(19) United States
(12) Patent Application Publication
(10) Pub. No.: US 2012/0208883 A1
(43) Pub. Date: Aug. 16, 2012

(54) TREATMENT OF ONCOLOGICAL DISEASES

(75) Inventors: Claudio Cavazza, Rome (IT); Anna Atti, Rome (IT); Enrico Cavazza, Rome (IT); Silvia Cavazza, Rome (IT); Francesca Cavazza, Rome (IT); Martina Cavazza, Rome (IT); Paolo Carminati, Milan (IT); Gabriella Singrossi, Milan (IT); Silvia Carminati, Milan (IT); Giuseppe Paolo Carminati, Milan (IT); Claudio Pisano, Aprilia (LT) (IT)

(73) Assignee: SIGMA-TAU INDUSTRIE FARMACEUTICHE RIUNITE S.P.A, Rome (IT)

(21) Appl. No.: 13/131,948

(22) PCT Filed: Dec. 1, 2009
(86) PCT No.: PCT/EP2009/066117

§ 371 Date: Apr. 9, 2012

Related U.S. Application Data
(60) Provisional application No. 61/248,543, filed on Oct. 5, 2009.

(30) Foreign Application Priority Data
Dec. 1, 2008 (EP) 08170331.6

(51) Int. Cl. A61K 31/221; A61P 35/02; A61P 35/00

(52) U.S. Cl. 514/547

(57) ABSTRACT

The present invention relates to the use of acetyl L-carnitine for the preparation of a medicament for the prevention and/or treatment of cancer. Methods of preventing and/or treatment of cancer by administering an effective amount of acetyl L-carnitine to subject in need thereof are also disclosed.

### FIELD OF THE INVENTION

The invention relates to a method of preventing or treating proliferative diseases or diseases that may be associated with or triggered by persistent angiogenesis in a mammal, particularly adult human, using acetyl L-carnitine in a dose higher than 0.5 g/day, preferably higher than 0.8 g/day; most preferably higher than 1 g/day.

### BACKGROUND OF THE INVENTION

Cancer is a class of diseases in which a group of cells display uncontrolled growth, invasion, and sometimes metastasis.

These three malignant properties of cancers differentiate them from benign cancers, which are self-limited, do not invade or metastasize.

Cancer may affect people at all ages, even fetuses, but the risk for most varieties increases with age. Cancer causes about 13% of all deaths. According to the American Cancer Society, 7.6 million people died from cancer in the world during 2007.

Most cancers can be treated and some cured, depending on the specific type, location, and stage. Once diagnosed, cancer is usually treated with a combination of surgery, chemotherapy and radiotherapy. As research develops, treatments are becoming more specific for different varieties of cancer.

The effectiveness of chemotherapy is often limited by toxicity to other tissues in the body. Radiation can also cause damage to normal tissue.

In the medical field, for treating cancer are widely used combinations of different chemotherapeutic agents. In fact most of the therapeutical protocols provide for the combined use of different antineoplastic agents; this procedure allows to enhance the treatment efficacy because the individual feedback to the agents can change according to the agent adopted.

The use of alkanoyl L-carnitines in the medical field is already known and their preparation process is described in U.S. Pat. No. 4,254,053.

In WO/2000/06134 the use of L-carnitine and its alkanoyl derivatives in the preparation of medicaments with anticancer activity is described. In particular in WO/2000/06134 the following data are reported:
Animals treated with vehicle alone and those treated with paclitaxel (taxol) in combination with acetyl L-carnitine: a statistically significant reduction of the tumour mass was found in the latter (see page 48, lines 16-19);
By contrast, comparison of the group treated with vehicle alone and the one treated with vehicle in combination with acetyl L-carnitine revealed no statistically significant differences in tumour mass growth at any of the observation times (page 48, lines 20-23);
Analysis of the data relating to the comparison between the group treated with paclitaxel (taxol) and the one treated with paclitaxel in combination with acetyl L-carnitine showed no significant differences in tumour weight (page 48, lines 23-26 and page 57, lines 1-7);
As regards the analysis of the number of metastases, the data obtained showed a statistically significant reduction in that number in the groups treated with paclitaxel, with paclitaxel in combination with acetyl L-carnitine and with vehicle in combination with acetyl L-carnitine as compared to the group treated with vehicle alone (page 49, lines 1-4);
In particular, the mice treated with paclitaxel or with paclitaxel in combination with acetyl L-carnitine also showed a reduction in the diameter of the metastases compared to the groups treated with vehicle alone or with vehicle in combination with acetyl L-carnitine (page 49, lines 4-8);
On the basis of analysis of the following data, it was therefore concluded that acetyl L-carnitine does not interfere with the anticancer action of paclitaxel in terms of inhibition of the tumour mass (page 49, lines 8-11);
In addition, acetyl L-carnitine (ALC) showed a significant inhibitory effect on the formation of lung metastases (page 49, lines 11-12);
Paclitaxel treatment caused an inhibition of tumour growth (TVI=88%). Treatment with ALC had no effect on tumour growth, which was similar to that in control group tumours. Combined treatment with paclitaxel plus ALC showed an anticancer efficacy (TVI=90%) almost identical to that achieved with paclitaxel alone, confirming that ALC did not interfere with the cytotoxic activity of paclitaxel (page 61 lines 4-9);
In the paclitaxel+propionyl L-carnitine (PLC) group versus the control group, with p<0.003, and only at the last observation time (day 46) did the significance level drop to p<0.034. It should be noted that the values for the paclitaxel group on day 46 were not significantly different from the control group values (page 66 last line and page 67 lines 1-4);
Only the control group was significantly different from the Paclitaxel+PLC group, with p<0.05 (page 67 last two lines).

It is important to note that in WO/2000/06134 ALC was administered orally at a dose of 100 mg/kg/mice. This does would correspond to a dose of about 0.5 g per day for administration to adult humans (see for example “Guidance for Industry and Reviewers; Estimating the Safe Starting Dose in Clinical Trials for Therapeutics in Adult Healthy Volunteers; Division of Drug Information, HFD-240; Center for Drug Evaluation and Research; Food and Drug Administration; 5600 Fishers Lane; Rockville, Md. 20857; http://www.fda.gov/cder/guidance/index.htm”—Table at page 233).

In Clinical Cancer Research Vol. 9; Nov. 15, 2003; p. 5756-5767; it is reported that ALC protects the mice from the lethal toxicity and from the neurotoxicity due to the use of the antitumor drug tested. About the antitumor activity in this publication it is reported that cisplatin alone significantly reduced the number of lung metastases and that the combination of ALC with cisplatin did not influence the antimetastatic or the antitumor effects of cisplatin.

It must be noted that the dose of ALC used in vivo (in mice) was of 100 mg/kg/day p.o. (which in adult human corresponds to about 0.5 g/day) and that the concentration of ALC used in vitro experiments was of 1 mM. It is also to be noted that the dose of cisplatin used in this paper ranges from 6 to 8 mg/kg (see Table 5).

In WO/2004/043454 the use of acetyl L-carnitine for the prevention and/or treatment of peripheral neuropathies induced by anticancer agents is described.

It is well-known that the use of anticancer agents in chemo therapy causes a large number of toxic or side effects which may lead to a reduction of the dose of the agent administered, and occasionally to discontinuation of the therapy itself. The reduction of the dose of the agent administered or the use of new anticancer agents not endowed with toxic side effects reduces the therapeutic efficacy of the anticancer agent.

Therefore the discovery of agents useful for increasing the pharmacological activity of anticancer agents or the use of new anticancer agents not endowed with toxic side effects remains a perceived need in the medical field.

Tumor protein p53 is a transcription factor that in humans is encoded by the TP53 gene. p53 is important in multicellular organisms, where it regulates the cell cycle and thus functions as a tumor suppressor that is involved in preventing cancer. This effect is observed with p53 from a variety of species, including humans, rodents, frogs, and fish. In a normal cell p53 is inactivated by its negative regulator, mdm2. Upon DNA damage or other stress, various pathways will lead to the dissociation of the p53 and mdm2 complex. Once activated, p53 will either induce a cell cycle arrest to allow repair and survival of the cell or apoptosis to discard the damage cell. How p53 makes this choice is currently unknown. p53 has many anticancer mechanisms, and plays a role in apoptosis, genetic stability, and inhibition of angiogenesis.

Mutant p53 can no longer bind DNA in an effective way, and as a consequence the p21 protein is not made available to act as the ‘stop signal’ for cell division. Thus cells divide uncontrollably, and form tumors. If the TP53 gene is damaged, tumor suppression is severely reduced. People who inherit only one functional copy of the TP53 gene will most likely develop tumors in early adulthood, a disease known as Li-Fraumeni syndrome. The TP53 gene can also be damaged in cells by mutagens (chemicals, radiation, or viruses), increasing the likelihood that the cell will begin decontrolled division. More than 50 percent of human tumors contain a mutation or deletion of the TP53 gene. Increasing the amount of p53, which may initially seem a good way to treat tumors or prevent them from spreading, is in actuality not a usable method of treatment, since it can cause premature aging.

However, restoring endogenous p53 function holds a lot of promise. In healthy humans, the p53 protein is continually produced and degraded in the cell. The degradation of the p53 protein is, as mentioned, associated with mdm2 binding. In a negative feedback loop mdm2 is itself induced by the p53 protein. However mutant p53 proteins often don't induce mdm2, and are thus able to accumulate at very high concentrations. Worse, mutant p53 protein itself can inhibit normal p53 protein levels.

### DESCRIPTION OF THE INVENTION

It has now been found that acetyl L-carnitine is an useful agent for the treatment or prevention of proliferative diseases or diseases that may be associated with or triggered by persistent angiogenesis, particularly neoplasms, in a mammal, particularly a human.

In particular, the invention described herein relates to methods of inhibition of the tumoral cell growth, a reduction of the tumoral cell mass, by administering an effective amount of acetyl L-carnitine or a pharmaceutically acceptable salt thereof to a subject in need thereof. The therapy can be combined with other known chemotherapies if desired for added clinical effectiveness.

What is meant by pharmaceutically acceptable salt of acetyl L-carnitine is any salt of the latter with an acid that does not give rise to toxic or side effects.

These acids are well known to pharmacologists and to experts in pharmacy. Non-limiting examples of such salts are: chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethanesulphonate, methanesulphonate, choline tartrate, trichloroacetate, and trifluoroacetate.

What is meant by pharmaceutically acceptable salt of L-carnitine is also a salt approved by the FDA and listed in the publication Int. J. of Pharm. 33 (1986), 201-217, the contents of which are incorporated herein by way of reference.

It is therefore an object of the present invention acetyl L-carnitine for use for reducing the tumour mass growth.

It is a further object of the present invention the use of acetyl L-carnitine for the preparation of a medicament for reducing the tumour mass growth.

According to the present invention the tumor is preferably a primary tumor.

It is a further object of the present invention the use of acetyl L-carnitine for the preparation of a medicament for reducing the tumour mass growth; in which the tumor cells have the p53 gene protein of wild type (not mutated).

It is a further object of the present invention the use of acetyl L-carnitine for the preparation of a medicament for reducing the tumour mass growth; in which the amount of acetyl L-carnitine to be administered (in human) is higher than 0.50 g/day, preferably higher than 0.8 g/day; most preferably higher than 1 g/day.

The pediatric dose may be subject to a reduction of one half or more.

This means that for administration to a pediatric patient the dose would typically be higher than 0.25 g/day, preferably higher than 0.4 g/day; most preferably higher than 0.5 g/day.

It is a further object of the present invention the use of acetyl L-carnitine for the preparation of a medicament for the inhibition of the tumour mass; in which the tumor cells contain the p53 gene protein of wild type (not mutated); and the amount of acetyl L-carnitine to be administered (in human) is higher than 0.50 g/day, preferably higher than 0.8 g/day; most preferably higher than 1 g/day.

Other and further objects including methods of treating or preventing one or more of the conditions including cancer are also provided in accordance with the present invention.

The Diseases to be Treated

The compound of the present invention is useful for treating proliferative diseases or diseases that are associated with or triggered by persistent angiogenesis, such as neoplasms.

The term “neoplasm” indicates an abnormal mass of tissue as a result of neoplasia. Neoplasia is the abnormal proliferation of cells. The growth of this clone of cells exceeds, and is uncoordinated with, that of the normal tissues around it. It usually causes a tumor. Neoplasms may be benign, pre-malignant or malignant:
benign neoplasms include for example uterine fibroids and melanocytic nevi. They do not transform into cancer.
potentially malignant neoplasms include carcinoma in situ. They do not invade and destroy but, given enough time, will transform into a cancer.
malignant neoplasms are commonly called cancer. They invade and destroy the surrounding tissue, may form metastases and eventually kill the host.

A primary tumor is a tumor growing at the anatomical site, where tumor progression began and proceeded to yield this mass.

Metastasis is the spread of a disease from one organ or part to another non-adjacent organ or part. Only malignant tumor cells and infections have the established capacity to metastasize. Cancer cells can break away, leak, or spill from a primary tumor, enter lymphatic and blood vessels, circulate through the bloodstream, and be deposited within normal tissue elsewhere in the body. Metastasis is one of three hallmarks of malignancy (contrast benign tumors). Most tumors and other neoplasms can metastasize, although in varying degrees (e.g., glioma and basal cell carcinoma rarely metastasize). When tumor cells metastasize, the new tumor is called a secondary or metastatic tumor, and its cells are like those in the original tumor.

According to an embodiment of the present invention the neoplasm to be treated is a primary tumor.

According to a further embodiment of the present invention the neoplasm to be treated is a malignant neoplasm, also called cancer, o a potentially malignant neoplasm.

The compound of the present invention is particularly useful for treating a cancer which is a breast cancer; lung cancer, including non-small cell lung cancer (NSCLC) and small-cell lung cancer (SCLC); gastrointestinal cancer, including esophageal, gastric, small bowel, large bowel, rectal and colon cancer; glioma, including glioblastoma; sarcoma, such as those involving bone, cartilage, soft tissue, muscle, blood and lymph vessels; ovarian cancer; myeloma; female cervical-cancer; endometrial cancer; head and neck cancer; mesothelioma; renal-cancer; uteran; bladder and urethral cancers; leukemia; lymphoma, prostate cancer; skin cancers; and melanoma. In particular, the inventive compositions are particularly useful for treating: i. a breast cancer; a lung cancer, e.g., non-small cell lung cancer, including non-small cell lung cancer (NSCLC) and small-cell lung cancer (SCLC); a gastrointestinal cancer, e.g., a colorectal cancer; or a genitourinary cancer, e.g., a prostate cancer; ovarian cancer; glioma, including glioblastoma; ii. a proliferative disease that is refractory to the treatment with other chemotherapeutics; or iii. a cancer that is refractory to treatment with other chemotherapeutics due to multidrug resistance.

In a broader sense of the invention, a proliferative disease may furthermore be a hyperproliferative condition, such as a leukemia, lymphoma or multiple myeloma. The combination of the present invention can also be used to prevent or treat diseases that are triggered by persistent angiogenesis, such as Kaposi's sarcoma, leukemia or arthritis.

The present invention also relates to the treatment of pediatric cancers.

An example of pediatric cancer that can be treated or inhibit the progress of the condition according to the present invention are selected from the group consisting of: acute lymphoblastic leukemia, acute myeloid leukemia, adrenocortical carcinoma, astrocytomas, bladder cancer, brain stem glioma, brain stem glioma, central nervous system atypical teratoid/rhabdoid cancer, brain cancer, central nervous system embryonal cancers, brain cancer, astrocytomas, craniopharyngioma, ependymoblastoma, ependymoma, childhood medulloblastoma, medulloepithelioma, pineal parenchymal cancers of intermediate differentiation, supratentorial primitive neuroectodermal cancers and pineoblastoma, breast cancer, bronchial cancers, carcinoid cancer, central nervous system atypical teratoid/rhabdoid cancer, central nervous system embryonal cancers, cervical cancer, chordoma, colorectal cancer, craniopharyngioma, ependymoblastoma, ependymoma, esophageal cancer, extracranial germ cell cancer, gastric cancer, glioma, hepatocellular (liver) cancer, hodgkin lymphoma, kidney cancer, laryngeal cancer, leukemia, acute lymphoblastic/myeloid leukemia, liver cancer, hodgkin lymphoma, non-hodgkin lymphoma, medulloblastoma, medulloepithelioma, mesothelioma, multiple endocrine neoplasia syndrome, acute myeloid leukemia, nasopharyngeal cancer, oral cancer, ovarian cancer, pancreatic cancer, papillomatosis, pineal parenchymal cancers of intermediate differentiation, pineoblastoma and supratentorial primitive neuroectodermal cancers, renal cell cancer, rhabdomyosarcoma, salivary gland cancer, sarcoma, skin cancer, gastric cancer, supratentorial primitive neuroectodermal cancers, thymoma and thymic carcinoma, thyroid cancer and vaginal cancer.

Where a cancer, a cancer disease, a carcinoma or a cancer are mentioned, also metastasis in the original organ or tissue and/or in any other location are implied alternatively or in addition, whatever the location of the cancer and/or metastasis.

The compositions are selectively toxic or more toxic to rapidly proliferating cells than to normal cells, particularly in human cancer cells, e.g., cancerous cancers, the compound has significant anti-proliferative effects and promotes differentiation, e.g., cell cycle arrest and apoptosis.

### DETAILED DESCRIPTION OF THE INVENTION

The invention is further defined by reference to the following examples. It will be apparent to those skilled in the art, that many modifications, both to materials, and methods, may be practiced with out departing from the purpose and interest of this invention. The examples that follow are not intended to limit the scope of the invention as defined hereinabove or as claimed below.

### Example 1

### Anti-Proliferative Activity In Vitro

The anti-proliferative activity of acetyl L-carnitine was evaluated in two different NSCLC tumor cell lines (NCI-H460 and NCI-H1650) and on one ovarian tumor cell line (A2780/Dx multidrug-resistant).

Cells were seeded in 96-well tissue culture plates and treated for three days (NCI-H460 NSCLC) or six days (NCI-H1650 NSCLC and A2780/Dx cells) with acetyl L-carnitine (10 mM in 0.1% FBS). To test the effect of acetyl L-carnitine on cell growth, tumor cells were seeded in 96-well tissue culture plates at approximately 10% confluence and were allowed to attach and recover for at least 24 h. Tumor cells were exposed to treatment protocol for 72 h or 6 days in 0.1% FBS at 37° C., then medium culture (RPMI 1640) was removed and 100 μl/well of medium were added containing 25 μl/well of a solution 5 mg/ml tetrazolium salt (MTT) assay, (final 1 mg/ml). Plates were kept at 37° C. in incubator with 5% CO2 for 2 h for the formation of blue crystals. The supernatant was removed and 100 μl/well of lysing medium were added. Plates were kept under stirring for 60 min. The cell survival was determined as optical density by a Multiskan spectrofluorimeter at 570 nm (Hansen M. B., et al., J. Immunol. Methods, 1989, 119, 203). Percentage of tumor cell inhibition in the presence of 10 mM acetyl L-carnitine was evaluated and compared with that obtained in the absence of acetyl L-carnitine applying the statistical comparison by means of the Mann-Whitney test.

The results obtained are reported in the following Tables 1-3.

TABLE 1
Activity on NCI-H460 cell line

| | Cell line NCI-H460 | | |
|---|---|---|---|
| | Absorbance ± | Tumor | |
| | SE | cell | |
| Treatment | at 570 nm | inhibition % | P value |
| Control | 745 ± 18 | / | / |
| Acetyl L- | 509 ± 8 | 32 | <0.001 |
| carnitine | | | |

On the tumoral line tested acetyl L-carnitine, at a concentration of 10 mM (72 h of treatment) was able to induce an antiproliferative effect which was statistically significant (32% of tumor cell inhibition).

TABLE 2
Activity on NCI-H1650 cell line

| | Cell line NCI-H1650 | | |
|---|---|---|---|
| | Absorbance ± | Tumor | |
| | SE | cell | |
| Treatment | at 570 nm | inhibition % | P value |
| Control | 484 ± 25 | / | / |
| Acetyl L- | 285 ± 11 | 41 | <0.001 |
| carnitine | | | |

On the tumoral line tested acetyl L-carnitine, at a concentration of 10 mM (6 days of treatment) was able to induce a statistically significant antiproliferative effect (41% of tumor cell inhibition).

TABLE 3
Activity on A2780/DX cell line

Cell line A2780/Dx MDR
ovarian ca.

| | Absorbance ± SE | Tumor cell |
|---|---|---|
| Treatment | at 570 nm | inhibition % |
| Control | 331 ± 18 | / |
| Acetyl L- | 234 ± 16 | 29 |
| carnitine | | |

On the tumoral line tested acetyl L-carnitine, at a concentration of 10 mM (6 days of treatment) was able to induce significant antiproliferative effect (29% of tumor cell inhibition).

### Example 2

Anti-tumoral activity in vivo (CD1 Nude mouse) using five different tumoral cell lines.

Lines tested:

NCI-H460 Cell Line

The tumors were generated by subcutaneous injection in the right flank of CD1 nude mice of NCI-H460 tumor cells (3×10⁶/100 μl/mouse), in 0.1 ml medium Tc199.

NCI-H460 Metastatic Clone

The tumors were generated by subcutaneous injection in the right flank of CD1 nude mice of NCI-H460 tumor cells (3×10⁶/100 μl/mouse), in 0.1 ml medium Tc199.

Human A549 NSCLC Cell Line

The tumors were generated by subcutaneous injection in the right flank of CD1 nude mice of A549 tumor cells (3×10⁶/100 μl/mouse) in 0.1 ml medium Tc199.

NCI-H1650 Cell Line

The tumors were generated by subcutaneous injection in the right flank of CD1 nude mice of NCI-H1650 tumor cells (5×10⁶/200 μl/mouse) in suspension in 0.1 ml medium Tc199+0.1 ml Matrigel.

A2780 ADR Cell Line

The tumors were generated by subcutaneous injection in the right flank of CD1 nude mice of A2780 ADR tumor cells (5×10⁶/100 μl/mouse) in 0.1 ml medium Tc199.

Treatments started when tumors were palpable, 8 mice per group were used.

Acetyl L-carnitine was administered po (oral administration) at 200 mg/kg according to the schedule qdx5/w (once per day for 5 days per week).

The reference compound cisplatin was administered ip (intra peritoneal) according to the schedule q4d/w, at the doses of 4 mg/kg or q7dx4 at the dose of 5 mg/kg.

The reference compound doxorubicin was delivered intravenously according to the schedule q7dx3, at a dose of 6 mg/kg.

Tumor volume (TV) was calculated biweekly by measuring the diameter of the tumors with a Vernier caliper according to the formula

TV=d²×D/2

where d and D are the shortest and longest diameters, respectively. The efficacy of the drug treatment was assessed as the tumor volume inhibition according to the formula reported underneath:

$$TVI \% = 100 - [ ( \frac{( mean TV of treated group}{( mean TV of control group} ) \times 100 ]$$

The results obtained are reported in the following Tables 4-8.

TABLE 4
NCI-H460 cell line
LINE NCI-H460

| COMPOUND | | Acetyl L-carnitine | Cisplatin |
|---|---|---|---|
| Dose (mg/kg)/ | | 200/po | 4/ip |
| route | | | |
| Schedule | | qdx5/wx4w | q4dx5 |
| Results | TVI % ± SE | 40 ± 6 | 41 ± 5 |

In this experimental model acetyl L-carnitine produced a statistically significant tumor volume inhibition comparable to that of the reference compound cisplatin.

Besides being unexpected, this was a very interesting result because acetyl L-carnitine is a non toxic compound not endowed with the toxic side effects of cisplatin.

TABLE 5
NCI-H460 metastatic clone
NCI-H460 metastatic clone

| | | Acetyl L- | |
|---|---|---|---|
| COMPOUND | | carnitine | Cisplatin |
| Dose (mg/kg)/route | | 200/po | 5/iv |
| Schedule | | qdx5/wx4w | q7dx4 |
| Results | TVI % | 34 ± 5 | 41 ± 5 |
| | % inhibition of | 50 | 0 |
| | lung metastases | | |

In this experimental model acetyl L-carnitine produced a statistically significant antimetastatic effect on spontaneous lung metastases induced by NCI-H460 metastatic clone injected subcutaneously, whereas cisplatin showed to be ineffective.

TABLE 6
Human A549 NSCLC cell line
Human A549 NSCLC cell line

| | | Acetyl L- | |
|---|---|---|---|
| COMPOUND | | carnitine | Cisplatin |
| Dose (mg/kg)/route | | 200/po | 4/ip |
| Schedule | | qdx5/wx4w | q4dx6 |
| Results | TVI % | 27 ± 3 | 49 ± 13 |

In this experimental model acetyl L-carnitine (respect to the negative control, data not shown) produced a statistically significant tumor volume inhibition (27%). This result is lower respect that obtained using a positive control, cisplatin (49%). Thanks to the absence of toxicity due to the use of acetyl L-carnitine these results are surprisingly and really interesting for the therapeutic use in the oncological field of this compound.

TABLE 7
NCI-H1650 cell line
TUMOR LINE NCI-H1650

| | | Acetyl L- | |
|---|---|---|---|
| COMPOUND | | carnitine | Cisplatin |
| Dose (mg/kg)/ | | 200/po | 4/ip |
| route | | | |
| Schedule | | qdx5/wx3w | q4dx6 |
| Results | TVI % | 19 ± 4 | 23 ± 3 |

In this experimental model acetyl L-carnitine produced a statistically significant tumor volume inhibition comparable to that of the reference compound cisplatin.

TABLE 8
A2780 ADR cell line
OVARIAN MULTIDRUG RESISTANT
LINE A2780-ADR

| | | Acetyl L- | |
|---|---|---|---|
| COMPOUND | | carnitine | Doxorubicin |
| Dose (mg/kg)/ | | 200/po | 6/iv |
| route | | | |
| Schedule | | qdx5/wx3w | q7dx3 |
| Results | TVI % | 28 ± 11 | 60 ± 11 |

In this experimental model acetyl L-carnitine (respect to the negative control, data not shown) produced a statistically significant tumor volume inhibition (28%).

This result was inferior to that obtained using a positive control, cisplatin (60%). Thanks to the absence of toxicity due to the use of acetyl L-carnitine this result is surprisingly and interesting for the therapeutic use of this compound.

### Example 3

Antiproliferative activity of acetyl-L-carnitine (10 mM) in comparison with cisplatin on the pediatric cancer SJSA-1 (with amplification of mdm2) osteosarcoma cells (72 h of exposure followed by 72 h of recovery).

The results obtained are reported in the following Table 9.

TABLE 9

| | Cell line SJSA-1 | | |
|---|---|---|---|
| | Absorbance | | P value |
| | % ± SE at | Tumor cell | VS |
| Treatment | 570 nm | inhibition % | Control |
| Control | 1306 ± 62 | / | / |
| (Cisplatin) | | | |
| Acetyl L- | **993 ± 46 | 24 | 0.0037 |
| carnitine | | | |

The results reported in Table 9 shown that acetyl L-carnitine is statistically significant more active than cisplatin when given chronically (72 h of exposure) to SJSA-1 osteosarcoma cells (pediatric cancer) cultured in medium containing 0.1% FBS.

The evaluation of antiproliferative activity was carried out by MTT assay.

### Example 4

Antiproliferative activity of L-carnitine in combination with cisplatin against NCI-H460 non-small cell lung carcinoma.
NCI-H460 cancer cells were inoculated subcutaneously (s.c.) in the right flank of CD1 nude mice (12 mice/group) (3×10⁶/100 μL/mouse). Treatments started three days after tumor injection.
To evaluate the antitumor activity, tumor diameters were measured with a Vernier caliper. The formula TV (mm³)=[length (mm)×width (mm)²]/2 was used, where the width and the length are the shortest and the longest diameters of each tumor, respectively and Log₁₀ cell kill (LCK) calculated by the formula LCK=(T−C)/3.32×DT, where T and C were the mean times (days) required for treated (T) and control (C) tumors, respectively, to reach 1 cm³, and DT was the doubling time of control tumors.
When tumors reached a volume of 1-2 cm³, mice were sacrificed by cervical dislocation. Body weight recording was carried out through the study and mortality was noted.
The results obtained are reported in the following Table 10.

TABLE 10
Antitumor activity of L-carnitine in combination with cisplatin against
NCI-H460 non-small cell lung carcinoma

| | Dose | | | | |
|---|---|---|---|---|---|
| | (mg/kg)/ | BWL % | | TV ± SE + | TVI % ± SE + |
| Treatment | route | max | Leth. | 32 | 32 |
| Vehicle | 0 | 0 | 0/8 | 1748 ± 273 | / |
| Cisplatin | 4/ip | 8 | 0/9 | 345 ± 90 | 80 ± 21 |
| L-carnitine + | 200/po + | 12 | 0/9 | 517 ± 68 | 70 ± 9 |
| cisplatin | 4/ip | | | | |

Tumor cells were inoculated at day 0. Treatment started on day +3 according to schedule qdx5/wx3w for L-carnitine and q4d/wx3w for cisplatin. DT = 3.8 days.

The results reported in Table 10 shown that L-carnitine at a dose of 200 mg/mice/day was not able to potentiate the cytotoxic activity of cisplatin when given chronically to NCI-H460 non-small cell lung carcinoma.

It is worthily to mention that the dose of ALC used (in mice) according to the present invention was of 200 mg/kg/day p.o. in vivo experiments, and 10 mM in vitro experiments. These doses are significantly higher than the dose of ALC used in the experiment reported in the references cited in the section Background of the Invention.

The composition according to the present invention comprises active ingredients which are known in the medical sector and already used in clinical practice. Therefore, they are very easy to procure, inasmuch as they are products which have been on the market for some time and are of a grade suitable for human or animal administration.

Acetyl L-carnitine is a known compound, which preparation process is described in U.S. Pat. No. 4,254,053. Acetyl L-carnitine may be administered in an amount higher than 0.50 g/day, preferably higher than 0.8 g/day; most preferably higher than 1 g/day to a mammal, preferably human, in need thereof. These dosage regimens can be continued for as long as clinically needed whether for the prevention or treatment of cancer. In these aspects where treatment is for reducing tumor cell mass or tumor cell growth or any other metastatic condition, the treatment amounts are the same as those set forth above. Interruptions or cycles of therapy commonly used with conventional chemotherapeutic agents are not required in view of the safety and exceptional patient tolerance of acetyl L-carnitine by patients.

The daily dose to be administered, according to the present invention will depend on the judgement of the primary care physician, on the subject's weight, age and general conditions.

The composition of the invention can have a form of tablet, sachet, capsule or vial, for oral, enteral or parenteral administration.

The pharmaceutical composition can be formulated supplying the component in a suitable liposome.

The compositions covered by the present invention are entirely conventional and are obtained with methods that are common practice in the pharmaceutical industry. According to the administration route opted for, the compositions will be in solid or liquid form, suitable for oral, parenteral or intravenous administration. The compositions according to the present invention contain, along with the active ingredient, at least one pharmaceutically acceptable vehicle or excipient. Particularly useful may be formulation adjuvants such as, for example, solubilising agents, dispersing agents, suspension agents and emulsifying agents. A general reference work is Remington's Pharmaceutical Sciences Handbook, latest edition.

1-12. (canceled)

13. A method of reducing a primary tumor mass growth in a human in need thereof comprising:
administering a medicament comprising an effective amount of acetyl L-carnitine or a pharmaceutically acceptable salt thereof to a human in need thereof; and
reducing a primary tumor mass growth in said human.

14. Method of claim 13, wherein said effective amount is higher than 0.5 g/day.

15. Method of claim 13, wherein said effective amount is higher than 0.8 g/day.

16. Method of claim 13, wherein said effective amount is higher than 1 g/day.

17. Method of claim 13, wherein the tumor cells comprise p53 gene protein wild type.

18. Method of claim 13, wherein the pharmaceutically acceptable salt acetyl L-carnitine is selected from the group consisting essentially of chloride, bromide, orotate, aspartate, acid aspartate, acid citrate, magnesium citrate, phosphate, acid phosphate, fumarate and acid fumarate, magnesium fumarate, lactate, maleate and acid maleate, oxalate, acid oxalate, pamoate, acid pamoate, sulphate, acid sulphate, glucose phosphate, tartrate and acid tartrate, glycerophosphate, mucate, magnesium tartrate, 2-amino-ethanesulphonate, magnesium 2-amino-ethanesulphonate, methanesulphonate, choline tartrate, trichloroacetate, and trifluoroacetate.

19. Method of claim 13, wherein the tumor is selected from the group consisting essentially of non-small cell lung cancer, small-cell lung cancer, gastrointestinal cancer, glioma, sarcoma, ovarian cancer, myeloma, female cervical-cancer, endometrial cancer, head and neck cancer, mesothelioma, renal cancer, uteran cancer, bladder and urethral cancers, leukemia, prostate cancer, skin cancers, melanoma, leukemia, lymphoma and multiple myeloma.

20. Method of claim 13, wherein the tumor is a pediatric tumor.

21. Method of claim 20, wherein the pediatric tumor is selected from the group consisting essentially of acute lymphoblastic leukemia, acute myeloid leukemia, adrenocortical carcinoma, astrocytomas, bladder cancer, brain stem glioma, central nervous system atypical teratoid/rhabdoid cancer, central nervous system embryonal cancers, brain cancer, craniopharyngioma, ependymoblastoma, ependymoma, childhood medulloblastoma, medulloepithelioma, pineal parenchimal cancers of intermediate differentiation, supratentorial primitive neuroectodermal cancers and pineoblastoma, breast cancer, bronchial cancers, carcinoid cancer, cervical cancer, chordoma, colorectal cancer, esophageal cancer, extracranial germ cell cancer, gastric cancer, glioma, hepatocellular (liver) cancer, Hodgkin lymphoma, kidney cancer, laryngeal cancer, leukemia, acute lymphoblastic/myeloid leukemia, liver cancer, non-hodgkin lymphoma, medulloblastoma, medulloepithelioma, mesothelioma, multiple endocrine neoplasia syndrome, acute myeloid leukemia, nasopharyngeal cancer, oral cancer, ovarian cancer, pancreatic cancer, papillomatosis, pineal parenchymal cancers of intermediate differentiation, pineoblastoma and supratentorial primitive neuroectodermal cancers, renal cell cancer, rhabdomyosarcoma, salivary gland cancer, sarcoma skin cancer, gastric cancer, thymoma and thymic carcinoma, thyroid cancer and vaginal cancer.

22. Method of claim 21, wherein the effective amount of acetyl L-carnitine is higher than 0.25 g/day.

23. Method of claim 21, wherein the effective amount of acetyl L-carnitine is higher than 0.4 g/day.

24. Method of claim 21, wherein the effective amount of acetyl L-carnitine is higher than 0.5 g/day.

25. Method of claim 13, wherein the acetyl L-carnitine is administered orally, parenterally, intravenously and/or transdermally.

26. Method of claim 13, wherein the acetyl L-carnitine is administered in a single dose schedule or in a multiple dose schedule.
